(19) United States
(12) Patent Application Publication
(10) Pub. No.: US 2008/0107713 A1
(43) Pub. Date: May 8, 2008

(54) Contact lens as a sustained drug delivery implant

(76) Inventors: Werhner C. Orilla, Anaheim, CA (US); James Burke, Santa Ana, CA (US); Michael Robinson, Irvine, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/594,518

(22) Filed: Nov. 8, 2006

(51) Int. Cl. A61K 9/00; B29D 11/00

(52) U.S. Cl. 424/429; 264/1.7

(57) ABSTRACT

A drug delivery device includes a contact lens having an optical axis and a plurality of discrete drug delivery zones asymmetrically disposed about the optical axis. A weighted area is provided in the contact lens for maintaining a presence of the contact lens upper hemispherical region in position with an upper hemispherical region of the user's eye. The asymmetrically disposed discrete drug delivery zones are positioned in the upper hemispherical region of the contact lens.

The present invention is generally related to a delivery vehicle for dispensing therapeutic drugs and is more particularly directed to a biocompatible contact lens incorporating drugs in a manner enabling a sustained and effective dose release through the cornea and other surrounding tissue.

Temporary and permanent disorders of the eye have been treated typically by disposing liquids, salves, or other medicaments directly in the eye by means of a squeeze tube or an eye dropper or flushing the eye with a wash glass.

In addition, “soft contact lenses” have been utilized as a delivery vehicle. Soft contact lenses are formed from a highly porous plastic which can absorb water or other fluids and accordingly by saturating the lens with a fluid medication and inserting the lens into the eye a dosage of medication is provided in the eye by the lacrimal fluids. Unfortunately, these simple devices do not provide a proper control sustained release of the medication.

Other soft contact lenses have utilized a polymeric plastic in which a reservoir or medication is held, for example, U.S. Pat. Nos. 3,618,604 and 3,828,777. In these devices, the polymeric material is designed to control the release rate of the medication and thus provide a more uniform level of medication within the eye for extended periods of time.

In order to maintain clarity of vision, other medication delivering contact lenses have been developed with transparent central optic areas and peripheral areas with a drug embedded therein as in U.S. Pat. No. 3,786,812.

However, none of these devices have provided for zone delivery of a drug which may be necessary in instances such as lowering of intra ocular pressure (IOP).

### SUMMARY OF THE INVENTION

A drug delivery device in accordance with the present invention generally may include a contact lens having an optical axis and a plurality of discrete drug delivery zones asymmetrically disposed of the optical axis. Preferably, the drug delivery zones are disposed in a spaced apart relationship with a central optical airing surrounding the optical axis and the drug delivery zones are disposed in an upper hemispherical region of a contact lens.

In addition, a weighted area may be provided in the contact lenses for maintaining a presence of the central lens upper hemispherical region in position with an upper hemispherical region of a users eye.

The delivery zones may be circular or oblate and in one embodiment of the present invention, a second contact lens is provided which conforms to the contact lens and the drug delivery zones are disposed or, sandwiched, between the contact lens and the second contact lens.

A method for delivery of a therapeutic drug to a user's eye in accordance with the present invention includes providing a contact lens having an optic axis for insertion by a user into a user's cornea and disposing a plurality of drug delivery zones asymmetrically about the optical axis and further instructing the user to install the contact lens into the user's cornea.

Additionally, in accordance with the present invention a method for fabricating a drug delivery device includes molding a contact lens from a non-hydrophilic material and disposing a drug on a concave surface of the contact lens. Thereafter, a second contact lens is molded onto the concave surface to sandwich the drug between the contact lens and the second contact lens. In this embodiment, the second contact lens is formed from a hydrophilic material.

### BRIEF DESCRIPTION OF THE DRAWINGS

The present invention may be more clearly understood with reference to the following detailed description, in connection with the appended drawings, in which:

FIG. 1 is a plan view of one embodiment of a contact lens in accordance with the present invention showing a plurality of discrete drug delivery zones asymmetrically disposed about an optical axis along with weighted areas;

FIG. 2 is a cross sectional view of the contact lens shown in FIG. 1 taken along the line 2-2;

FIG. 3 is a plan view of an alternative embodiment in accordance with the present invention also illustrating a plurality of discrete drug delivery zones asymmetrically disposed about an optical axis along with a weighted area; and

FIG. 4 is a cross sectional view of the contact lens shown in FIG. 3 taken along the line 4-4 and also showing a second contact lens conforming to a concave surface of the contact lens.

### DETAILED DESCRIPTION

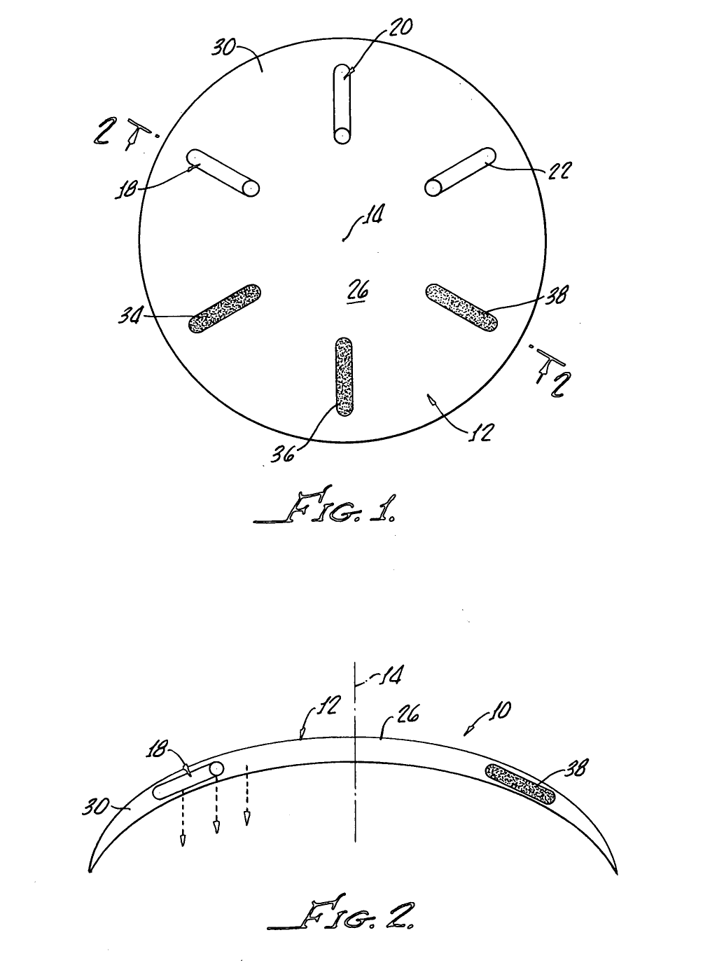

With reference to FIG. 1, there is shown a drug delivery device 10 in accordance with the present invention which generally includes a contact lens 12 having an optical axis 14. The lens 12 may cover the cornea and/or the peri-limbar area (not shown) and may be soft or hard utilizing silicone or any suitable commercial contact lens formulation.

A plurality of drug delivery zones 18, 20, 22 are asymmetrically disposed about the optical axis 14 generally in a spaced apart relationship with a central optical area 26. Any number of drug delivery zones 18, 20, 22 may be utilized with only three being shown in the embodiment 10 for illustrating the structure of the present invention.

The zones 18, 20, 22 may be reservoirs for therapeutic drugs or the drugs may be incorporated into a PLA+PLGA matrix/microspheres zones allowing a controlled sustained release illustrated by the arrows 30 in FIG. 2. Such reservoir enable the use of higher concentrated active agents.

The zones 18, 20, 22 may be of any suitable shape arid include, for example, any interocular pressure (IOP) lowering drug such as, for example, Brimo Tartrate+DDS PLA/PLGA rods.

The zones 18, 20, 22 are preferably disposed in a upper hemispherical region 30 for the purpose of avoiding the conjunctive, enhancing downward draining of the released drug, reducing wash and providing greater response of the eye to the drug.

In order to maintain a presence of the contact lens 12 upper hemispherical region 30 in position with an upper hemispherical region of a user's eye (not shown) weighted areas 34, 36, 38 are provided. It may be appreciated at any configuration of suitable weights including shape, size, and number may be utilized in the weighted areas or regions 34, 36, 38.

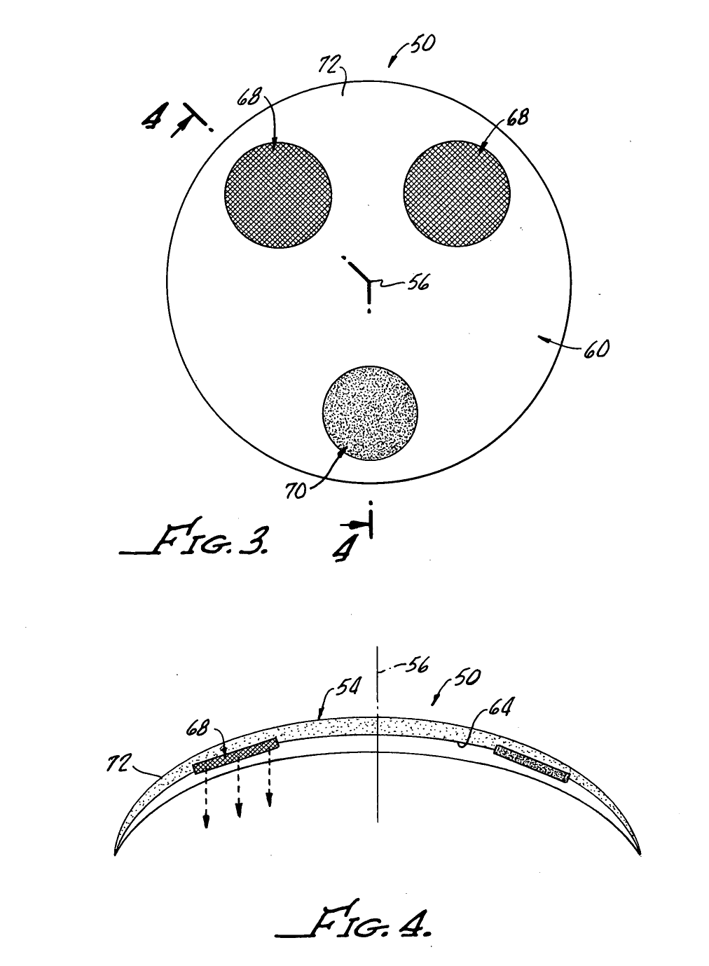

With reference to FIGS. 3 and 4, there is shown an alternative drug delivery device 50 in accordance with the present invention which includes a contact lens 54 having an optical axis 56 along with a second contact lens 60, as shown in FIG. 4, conforming to a concave surface 64 of a contact lens 54. A plurality of discrete drug delivery zones 68, 70 are asymmetrically disposed about the optical axis 56 and, as best shown in FIG. 4, between the contact lens 54 and the second contact lens 60. In this embodiment, the first contact lens 54 is an outer layer of non-hydrophilic material whereas the second contact lens 60 or inner layer, is a hydrophilic contact lens.

The zones 68, 70 may include Brimo Tartrate+DDS PLA/PLGA disc wafers which are sandwiched between the contact lens 54 and second contact lens 60. As with the embodiment 10, various shapes and numbers of drug delivery zones 68, 70 may be shown with two circular zones being shown for illustrative purposes only. The non-hydrophilic nature of the contact lens 54, or outer layer, prevents drug from leaking forward and the second contact lens 60, or inner layer, is hydrophilic which enables a drug to pass inwardly toward the eye (not shown).

As in the embodiment 10 a weighted area 70 maintains the orientation of the device 50 so that a presence of a contact lens upper hemispherical region 72 is in position with an upper hemispherical region of a user's eye (not shown).

A method for delivering a therapeutic and drug to a user's eye in accordance with the present invention includes providing a contact lens 10 having an optic axis 14 for insertion by a user into a user's cornea (not shown), disposing a plurality of drug delivery zones 18, 20, 22 asymmetrically about the optic axis 14 and instructing the user to install a contact lens 12 onto the user's cornea.

Further, a method for fabricating a drug delivery device 50 includes molding a contact lens 54 from a non-hydrophilic material, disposing a drug on a concave surface 64 of a contact lens 54 and thereafter molding a second contact lens 60 onto the concave surface 64 to sandwich the drug between the contact lens 54 and the second contact lens 60, the second contact lens being formed from a hydrophilic material.

Suitable non-hydrophilic materials include silicones such as unrestricted platinum fast-cure Nusil MED1-4213 and MED2-4123 and unrestricted Nusil low-and high-consistency elastomers with platinum cure systems. Alternatively, medical grade silicones commercially available may also be used as well as conventional hydrogel polymers.

Hydrophilic materials suitable for use in the present invention include: lower water content, non-ionic polymers (e.g. crofilcon); high water content, non-ionic polymers (e.g. lidofilcon; lower water content, ionic polymers (e.g. balafilcon); and high water content, ionic polymers (e.g. etafilcon A).

Although there has been hereinabove described a specific contact lens as a sustained drug delivery implant in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

1. A drug delivery device comprising:
a contact lens having an optical axis; and
a plurality of discrete drug delivery zones asymmetrically disposed about said optical axis.

2. The device according to claim 1 wherein said drug delivery zones are disposed in a spaced apart relationship with a central optical area surrounding said optical axis.

3. The device according to claim 2 wherein said drug delivery zones are disposed in an upper hemispherical region of said contact lens.

4. The device according to claim 3 further comprising a weighted area in said contact lens for maintaining a presence of the contact lens upper hemispherical region in position with an upper hemispherical region of a users' eye.

5. The device according to any one of claims 1-4 wherein said drug delivery zones are circular.

6. The device according to any one of claims 1-4 wherein said drug delivery zones are oblate.

7. The device according to claim 1 further comprising a second contact lens conforming to the contact lens and said drug delivery zones are disposed between said contact lens and said second contact lens.

8. The device according to claim 7 wherein said drug delivery zones are disposed in a spaced apart relationship with a central optical area surrounding said optical axis.

9. The device according to claim 8 wherein said drug delivery zones are disposed in an upper hemispherical region of said contact lens.

10. The device according to claim 9 further comprising a weighted area in said contact lens for maintaining a presence of the contact lens upper hemispherical region in position with an upper hemispherical region of a users' eye.

11. The device according to any one of claims 7-10 wherein said drug delivery zones are circular.

12. The device according to any one of claims 7-10 wherein said drug delivery zones are oblate.

13. A drug delivery device comprising:
a contact lens having an optical axis;
a second contact lens conforming to said contact lens; and
a plurality of discrete drug delivery zones asymmetrically disposed about said optical axis and between the contact lens and the second contact lens.

14. The device according to claim 13 wherein said drug delivery zones are disposed in a spaced apart relationship with a central optical area surrounding said optical axis.

15. The device according to claim 14 wherein said drug delivery zones are disposed in an upper hemispherical region of said contact lens.

16. The device according to claim 15 further comprising a weighted area in said contact lens for maintaining a presence of the contact lens upper hemispherical region in position with an upper hemispherical region of a users' eye.

17. A method for delivery a therapeutic drug to a user's eye comprising:
providing a contact lens, having an optic axis, for insertion by a user into a user's cornea;
disposing a plurality of drug delivery zones asymmetrically about the optic axis; and
instructing said user to install the contact lens onto the user's cornea.

18. The method according to claim 17 further comprising providing a contact lens with drug delivery zones disposed in a spaced apart relationship with a central optical area surrounding said optical axis.

19. The method according to claim 18 further comprising providing a contact lens with delivery zones in an upper hemispherical region of said contact lens.

20. The method according to claim 19 further comprising providing a contact lens with a weighted area for maintaining a presence of the contact lens upper hemispherical region with an upper hemispherical region of the user's eye.

21. A method for fabricating a drug delivery system comprising:
molding a contact lens from a non-hydrophilic material;
disposing a drug as a concave surface of said contact lens; and
molding a second contact lens onto said concave surface to sandwich said drug between said contact lens and said second contact lens, said second contact lens being found from a hydrophilic material.
